(19) United States
(12) Patent Application Publication
(10) Pub. No.: US 2021/0063340 A1
(43) Pub. Date: Mar. 4, 2021

(54) CHIP, METHOD OF OPERATING CHIP, AND DETECTION DEVICE

(71) Applicants: BEIJING BOE OPTOELECTRONICS TECHNOLOGY CO., LTD., Beijing (CN); BOE TECHNOLOGY GROUP CO., LTD., Beijing (CN)

(72) Inventors: Wenliang YAO, Beijing (CN); Haochen CUI, Beijing (CN); Peizhi CAI, Beijing (CN); Fengchun PANG, Beijing (CN); Yue GENG, Beijing (CN); Le GU, Beijing (CN); Nan ZHAO, Beijing (CN); Yuelei XIAO, Beijing (CN); Hui LIAO, Beijing (CN); Yingying ZHAO, Beijing (CN); Chuncheng CHE, Beijing (CN)

(73) Assignees: BEIJING BOE OPTOELECTRONICS TECHNOLOGY CO., LTD., Beijing (CN); BOE TECHNOLOGY GROUP CO., LTD., Beijing (CN)

(21) Appl. No.: 16/632,661

(22) PCT Filed: Jan. 2, 2019
(86) PCT No.: PCT/CN2019/070122

§ 371 Date: Jan. 21, 2020

(51) Int. Cl. G01N 27/327; B01L 3/00; H01H 29/28

(52) U.S. Cl. CPC G01N 27/3271; H01H 29/28; B01L 3/50273

(57) ABSTRACT

A chip, a method of operating a chip, and a detection device are provided. The chip includes a detection cavity and a working electrode, the detection cavity is configured to be capable of containing a plurality of droplets, and the working electrode is arranged in the detection cavity and is configured to regularly arrange the plurality of droplets in the detection cavity along an extending direction of the working electrode.

### TECHNICAL FIELD

Embodiments of the present disclosure relate to a chip, a method of operating a chip, and a detection device.

### BACKGROUND

In biomedical tests, target nucleic acid sequences (such as particular deoxyribonucleic acid (DNA) fragments) are often present in complex mixtures (such as cell extracts) and are low in content. Therefore, when detecting particular DNA fragments or microorganisms in this complex mixture, base complementary hybridization appears to be ineffective.

Polymerase chain reaction (PCR) is a molecular biology technology used to amplify particular DNA fragments. The PCR technology uses a DNA fragment as a template, and amplifies (i.e., replicates) the DNA fragment to a sufficient number with the cooperation of DNA polymerases and nucleotide substrates. Therefore, the PCR technology can expand the number of the particular DNA fragments by several orders of magnitude, so as to facilitate subsequent qualitative or quantitative research and analysis of the DNA fragments based on the principle of base complementary hybridization using probes.

### SUMMARY

At least one embodiment of the present disclosure provides a chip, which includes: a detection cavity, configured to be capable of containing a plurality of droplets; and a working electrode, arranged in the detection cavity and configured to regularly arrange the plurality of droplets in the detection cavity along an extending direction of the working electrode.

For example, in the chip according to an embodiment of the present disclosure, the working electrode comprises a first electrode and a second electrode, the first electrode comprises a plurality of first comb portions, the second electrode comprises a plurality of second comb portions, and the plurality of first comb portions of the first electrode and the plurality of second comb portions of the second electrode are arranged in an interlaced manner.

For example, in the chip according to an embodiment of the present disclosure, each of the plurality of first comb portions and the plurality of second comb portions is of a straight line shape, a polyline shape, or a curve shape.

For example, in the chip according to an embodiment of the present disclosure, the plurality of first comb portions and the plurality of second comb portions are arranged side by side and are equally spaced.

For example, in the chip according to an embodiment of the present disclosure, the detection cavity comprises a liquid inlet and a liquid outlet, and the liquid inlet and the liquid outlet are at opposite ends of the detection cavity in the extending direction of the working electrode.

For example, the chip according to an embodiment of the present disclosure further includes a droplet generation structure, and the droplet generation structure is connected with the liquid inlet, and is configured to generate the plurality of droplets and input the plurality of droplets into the detection cavity through the liquid inlet.

For example, in the chip according to an embodiment of the present disclosure, the droplet generation structure comprises a first entry, a second entry, a first channel, a second channel, and a cross connection portion, the first entry is configured to be in communication with the first channel, the second entry is configured to be in communication with the second channel, the first channel and the second channel are connected through the cross connection portion, and the cross connection portion is connected with the liquid inlet.

For example, the chip according to an embodiment of the present disclosure further includes a first substrate, a second substrate, and a spacer layer, the chip further comprises an injection area and a detection area, the spacer layer defines the droplet generation structure in the injection area, and the spacer layer defines the detection cavity in the detection area.

For example, in the chip according to an embodiment of the present disclosure, the working electrode is on a side of the first substrate close to the second substrate or on a side of the second substrate close to the first substrate.

For example, in the chip according to an embodiment of the present disclosure, at least one of the first substrate and the second substrate is a transparent substrate.

For example, the chip according to an embodiment of the present disclosure further includes a passageway, and the passageway is in the detection cavity and arranged along the extending direction of the working electrode.

For example, in the chip according to an embodiment of the present disclosure, the spacer layer further defines the passageway in the detection area, a part of the spacer layer defining the passageway serves as a side wall of the passageway, the side wall is on the first comb portions and/or the second comb portions, and the passageway comprises a space corresponding to edges, which are close to each other, of two adjacent first comb portions, two adjacent second comb portions, or a first comb portion and a second comb portion that are adjacent.

At least one embodiment of the present disclosure further provides a detection device, which includes the chip according to any one of the embodiments of the present disclosure, and further includes a control unit, and the control unit is configured to apply an electrical signal to the working electrode.

For example, in the detection device according to an embodiment of the present disclosure, the electrical signal comprises an alternating electrical signal.

At least one embodiment of the present disclosure further provides a method of operating the chip according to any one of the embodiments of the present disclosure, and the method includes: applying an electrical signal to the working electrode to regularly arrange the plurality of droplets contained in the detection cavity along the extending direction of the working electrode.

For example, the method of operating the chip according to an embodiment of the present disclosure further includes: injecting a continuous phase and a dispersed phase into a droplet generation structure, so as to generate the plurality of droplets and enable the plurality of droplets to enter the detection cavity.

For example, in the method of operating the chip according to an embodiment of the present disclosure, a first entry of the droplet generation structure is configured to receive the continuous phase, and a second entry of the droplet generation structure is configured to receive the dispersed phase.

For example, in the method of operating the chip according to an embodiment of the present disclosure, the continuous phase comprises a mineral oil, and the dispersed phase comprises a sample solution.

For example, in the method of operating the chip according to an embodiment of the present disclosure, the continuous phase and the dispersed phase are injected by using an air pressure injection method.

For example, the method of operating the chip according to an embodiment of the present disclosure further includes: enabling the plurality of droplets contained in the detection cavity to perform an amplification reaction.

### BRIEF DESCRIPTION OF THE DRAWINGS

In order to clearly illustrate the technical solution of the embodiments of the present disclosure, the drawings of the embodiments will be briefly described in the following; it is obvious that the described drawings are only related to some embodiments of the present disclosure and thus are not limitative of the present disclosure.

FIG. 1A is a schematic plane diagram of a chip according to an embodiment of the present disclosure;

FIG. 1B is a schematic cross-sectional diagram of a chip according to an embodiment of the present disclosure along an N-N′ direction in FIG. 1A;

FIG. 2A is a schematic plane diagram of a chip according to an embodiment of the present disclosure in a state where no electrical signal is applied;

FIG. 2B is a schematic cross-sectional diagram of a chip according to an embodiment of the present disclosure in a state where no electrical signal is applied;

FIG. 3A is a schematic plane diagram of a chip according to an embodiment of the present disclosure in a state where an electrical signal is applied;

FIG. 3B is a schematic cross-sectional diagram of a chip according to an embodiment of the present disclosure in a state where an electrical signal is applied;

FIG. 4 is a schematic cross-sectional diagram of another chip according to an embodiment of the present disclosure along an N-N′ direction in FIG. 1A;

FIG. 5 is a schematic block diagram of a detection device according to an embodiment of the present disclosure;

FIG. 6 is a schematic flow chart of a method of operating a chip according to an embodiment of the present disclosure; and

FIG. 7 is a schematic flow chart of another method of operating a chip according to an embodiment of the present disclosure.

### DETAILED DESCRIPTION

In order to make objects, technical details and advantages of the embodiments of the present disclosure apparent, the technical solutions of the embodiments will be described in a clearly and fully understandable way in connection with the drawings related to the embodiments of the present disclosure. Apparently, the described embodiments are just a part but not all of the embodiments of the present disclosure. Based on the described embodiments herein, those skilled in the art can obtain other embodiment(s), without any inventive work, which should be within the scope of the present disclosure.

Unless otherwise defined, all the technical and scientific terms used herein have the same meanings as commonly understood by one of ordinary skill in the art to which the present disclosure belongs. The terms “first,” “second,” etc. which are used in the description and the claims of the present application for invention, are not intended to indicate any sequence, amount or importance, but distinguish various components. Also, the terms such as “a,” “an,” etc., are not intended to limit the amount, but indicate the existence of at least one. The terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly. “On,” “under.” “right,” “left” and the like are only used to indicate relative position relationship, and when the position of the object which is described is changed, the relative position relationship may be changed accordingly.

In the PCR technology, after an amplification operation is performed on a DNA fragment, a qualitative or quantitative detection of DNA fragments is required. A digital PCR detection technology uses an absolute quantitative method to directly detect the number of copies of the DNA fragment, and has higher sensitivity and specificity compared with a traditional fluorescent quantitative PCR detection technology. In the digital PCR detection process, droplets are usually tiled randomly in a micro-cavity and an image of the entire area is captured by, for example, a charge coupled device (CCD), and then obtained images are stitched and processed to obtain the number of copies of the DNA fragments through the fluorescence signal information of each droplet.

However, the randomness in tiling the droplets places high requirements on optical imaging and image analysis, which increases the costs of instruments and prolongs the detection time. Meanwhile, problems such as overlapping and inconsistent focal planes occurred in the droplet tiling process also adversely affect the precision and accuracy of the detection result.

At least one embodiment of the present disclosure provides a chip, a method of operating a chip, and a detection device. The chip can arrange droplets regularly, realize three-dimensional control of droplet positions, reduce the complexity and costs of an optical system for detection, reduce the difficulty of image processing, improve detection accuracy and detection efficiency, achieve miniaturization and integration of detection chips, and reduce manual operations.

Hereinafter, embodiments of the present disclosure will be described in detail with reference to accompanying drawings. It should be noted that the same reference numeral in different drawings is used to refer to the same element that has been described.

At least one embodiment of the present disclosure provides a chip, which includes a detection cavity and a working electrode. The detection cavity is configured to be capable of containing a plurality of droplets, and the working electrode is arranged in the detection cavity and is configured to regularly arrange the plurality of droplets in the detection cavity along an extending direction of the working electrode.

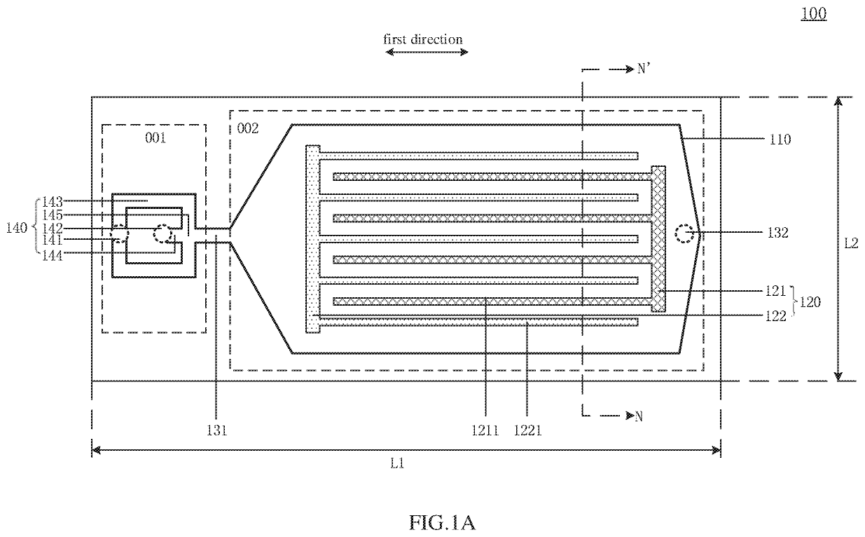

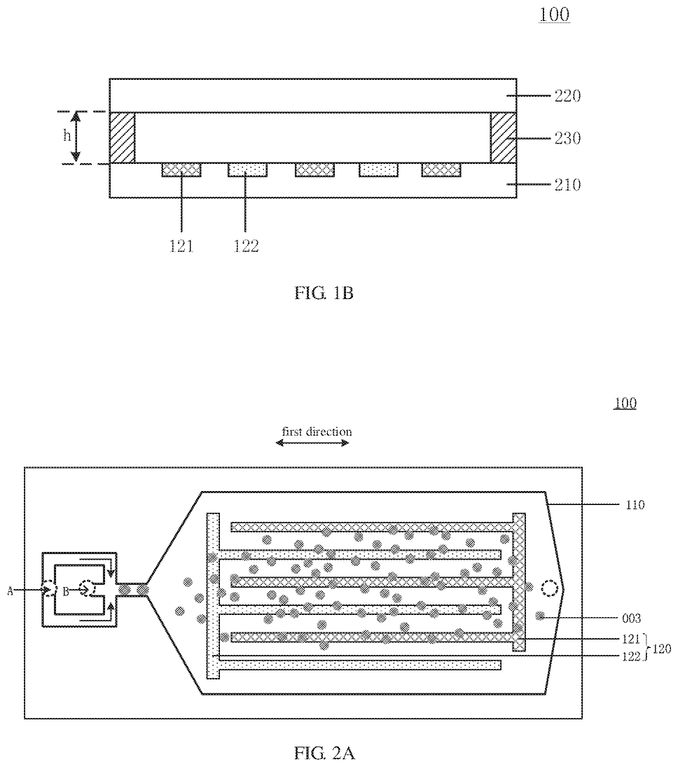

FIG. 1A is a schematic plane diagram of a chip according to an embodiment of the present disclosure. As illustrated in FIG. 1A, a chip 100 includes a detection cavity 110 and a working electrode 120.

The detection cavity 110 is configured to be capable of containing a plurality of droplets. For example, the plurality of droplets is a PCR reaction solution which includes to-be-detected DNA fragments on which an amplification reaction has been performed. The plurality of droplets are used for subsequent optical detection, for example, imaging using a CCD and subsequent image stitching and image processing, etc., thereby obtaining the number of copies of the DNA fragments through the fluorescence signal information of each droplet. For example, the detection cavity 110 is filled with oil phase substances (for example, a mineral oil), and the plurality of droplets are distributed in the mineral oil to be separated from each other and to be capable of moving freely. For example, the mineral oil may also include appropriate additives to improve the droplet wrapping property.

For example, the detection cavity 110 includes a liquid inlet 131 and a liquid outlet 132, the liquid inlet 131 is used to input the droplets into the detection cavity 110, and the liquid outlet 132 is used to discharge the droplets from the detection cavity 110. For example, as illustrated in FIG. 1A, the liquid inlet 131 and the liquid outlet 132 are arranged at opposite ends of the detection cavity 110 in an extending direction of the working electrode 120. The extending direction of the working electrode 120 is described hereinafter, and thus is not described in detail here. It should be noted that the liquid inlet 131 and the liquid outlet 132 are not limited to be arranged at the positions illustrated in FIG. 1A, and may be arranged at other appropriate positions, which is not limited in the embodiments of the present disclosure.

For example, in an example, the plurality of droplets may enter the detection cavity 110 through the liquid inlet 131 before subjected to the amplification reaction, and the amplification reaction may be carried out in the detection cavity 110 by using an additionally provided temperature control device. For example, in another example, the amplification reaction may be performed on the plurality of droplets in an additionally provided device, and then the plurality of droplets enter the detection cavity 110 through the liquid inlet 131 for detection. The embodiments of the present disclosure have no limitation in this aspect.

The working electrode 120 is arranged in the detection cavity 110 and is configured to regularly arrange the plurality of droplets in the detection cavity 110 along the extending direction of the working electrode 120. For example, the working electrode 120 includes a first electrode 121 and a second electrode 122. The first electrode 121 includes a plurality of first comb portions 1211, the second electrode 122 includes a plurality of second comb portions 1221, the first comb portions 1211 of the first electrode 121 and the second comb portions 1221 of the second electrode 122 are arranged in an interlaced manner. For example, in an example, the first electrode 121 and the second electrode 122 are conventional planar interdigital electrodes.

For example, the first electrode 121 and the second electrode 122 are electrically connected with an additionally provided control unit or control circuit (not illustrated in the figure), and the control unit or the control circuit is configured to apply an electrical signal (for example, an alternating electrical signal) to the first electrode 121 and the second electrode 122, so as to generate a non-uniform electric field in the detection cavity 110. The droplets contained in the detection cavity 110 are polarized under the action of the non-uniform electric field, so as to move to edges of the first comb portions 1211 and the second comb portions 1221 under the action of a dielectrophoresis force, thereby enabling the plurality of droplets to be regularly arranged in the extending direction of the working electrode 120. Here, the extending direction of the working electrode 120 refers to the extending direction of the first comb portion 1211 and the second comb portion 1221, i.e., a first direction illustrated in FIG. 1A.

For example, the first comb portions 1211 and the second comb portions 1221 may be of a straight line shape, a polyline shape (for example, a Z shape), a curve shape (for example, a wave shape), or other appropriate shape, as long as the first comb portions 1211 and the second comb portions 1221 are arranged in an interlaced manner, and the embodiments of the present disclosure have no limitation in this aspect. For example, in an example, the first comb portions 1211 and the second comb portions 1221 are arranged side by side and are equally spaced, so that the plurality of droplets may be uniformly arranged, thereby facilitating subsequent optical detection and helping to improving detection accuracy.

For example, the chip 100 also includes a droplet generation structure 140. The droplet generation structure 140 is connected with the liquid inlet 131, and is configured to generate the plurality of droplets and input the plurality of droplets into the detection cavity 110 through the liquid inlet 131. For example, in an example, the droplet generation structure 140 includes a first entry 141, a second entry 142, a first channel 143, a second channel 144, and a cross connection portion 145. The first entry 141 is configured to be in communication with the first channel 143, the second entry 142 is configured to be in communication with the second channel 144, the first channel 143 and the second channel 144 are connected to each other through the cross connection portion 145, and the cross connection portion 145 is connected with the liquid inlet 131. For example, in an example, a continuous phase (for example, a mineral oil) is input into the first entry 141, a dispersed phase (for example, a PCR reaction solution) is input into the second entry 142, and a speed gradient is generated in the PCR reaction solution by means of the interaction of a shear force, a viscous force and a surface tension generated by the collision and mixing of the two fluids, so as to achieve the wrapping and dividing of the PCR reaction solution, thereby generating the plurality of liquid droplets and enabling the plurality of droplets to enter the detection cavity 110 through the liquid inlet 131.

It should be noted that in the embodiments of the present disclosure, the droplet generation structure 140 is not limited to the above-mentioned structure, and may be a micro channel structure such as a conventional T-shaped structure, a conventional Y-shaped structure in the microfluidic chip, or other flow-focusing micro channel structure, and the embodiments of the present disclosure have no limitation in this aspect. In addition, the inputting manner and inputting position of the continuous phase and the dispersed phase through the entries are not limited, and may be determined based on actual requirements.

FIG. 1B is a schematic cross-sectional diagram of a chip according to an embodiment of the present disclosure along an N-N′ direction in FIG. 1A. As illustrated in FIG. 1B, the chip 100 may further include a first substrate 210, a second substrate 220, and a spacer layer 230.

The first substrate 210 and the second substrate 220 play a role of supporting, protecting, and the like, and are used to provide a space for accommodating the droplets. For example, the first substrate 210 and the second substrate 220 may be combined at the edge positions of the first substrate 210 and the second substrate 220 by using materials such as a sealant and an optical adhesive, and a certain space may be reserved between the first substrate 210 and the second substrate 220. For example, the first substrate 210 and/or the second substrate 220 may be a plastic substrate, a glass substrate, a silicon substrate, or other appropriate substrates, which is not limited in the embodiments of the present disclosure. For example, in a case where the glass substrate is adopted, the cost is lower, and in a case where the silicon substrate is adopted, the performance is better.

For example, at least one of the first substrate 210 and the second substrate 220 is a transparent substrate, for example, a glass substrate, a plastic substrate, etc. Because at least one of the first substrate 210 and the second substrate 220 is a transparent substrate, in a case where the droplets are regularly arranged and the optical detection is performed, the fluorescence generated by the droplets can pass through the transparent substrate with no loss or a low loss, so that the accuracy of the optical detection is improved and the requirements on the additionally provided optical detection devices are lowered.

For example, the chip 100 further includes an injection area 001 and a detection area 002 as illustrated in FIG. 1A, the spacer layer 230 defines the droplet generation structure 140 in the injection area 001, and the spacer layer 230 defines the detection cavity 110 in the detection area 002. For example, the spacer layer 230 may be made of any appropriate materials such as an optical adhesive, a resin, and the like, and may be formed for example in the form of a division wall. For example, the spacer layer 230 may be obtained by using wet etching or other appropriate processes.

For example, the working electrode 120 (i.e., the first electrode 121 and the second electrode 122) is arranged on a side of the first substrate 210 close to the second substrate 220, or on a side of the second substrate 220 close to the first substrate 210. For example, in an example, the working electrode 120 is arranged on a side of the first substrate 210 close to the second substrate 220, and the second substrate 220 is a transparent substrate. Therefore, during the optical detection, it is possible to prevent the working electrode 120 from blocking the fluorescence generated by the droplets and passing through the second substrate 220, which is conductive to improving the precision and accuracy of the optical detection and lowers the requirements on the material of the working electrode 120. In addition, it is possible for the working electrode 120 to adopt a transparent conductive material or a lightproof metal material. For example, the working electrode 120 may be made of a transparent conductive material such as indium tin oxide (ITO) or indium zinc oxide (IZO), or a metal material such as molybdenum or chromium, which is not limited in the embodiments of the present disclosure. For example, the working electrode 120 may be obtained by using a sputtering process, an etching process, or the like.

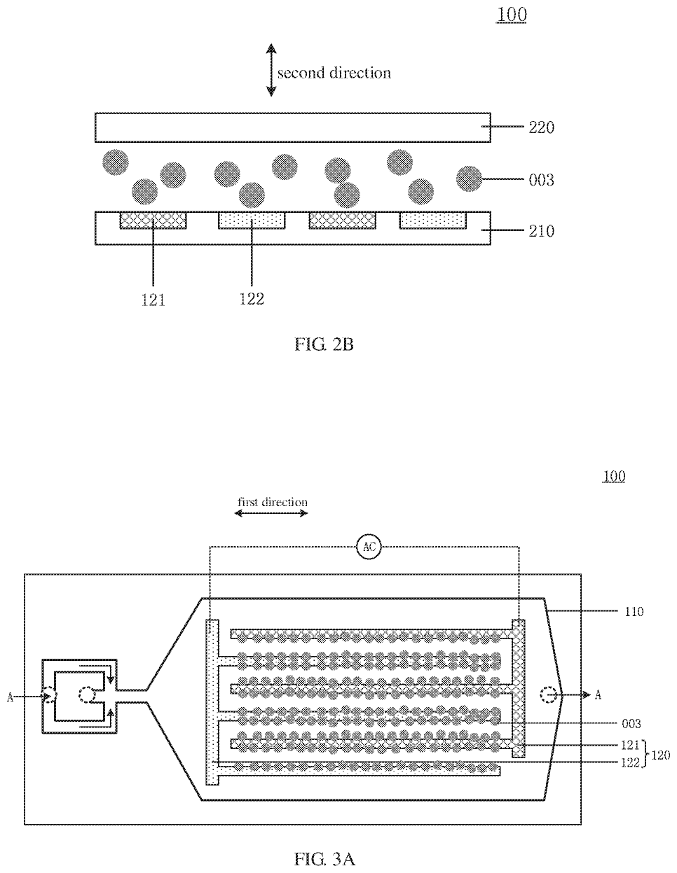

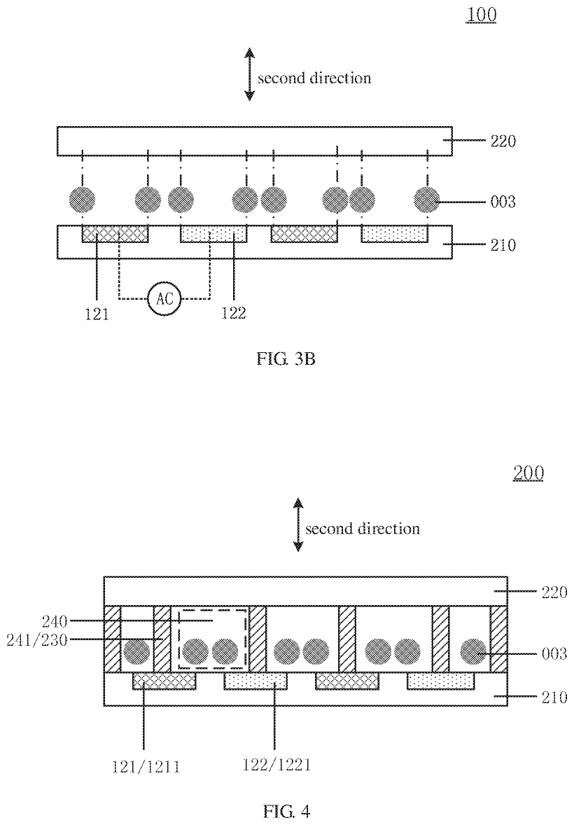

FIG. 2A is a schematic plane diagram of a chip according to an embodiment of the present disclosure in a state where no electrical signal is applied; FIG. 2B is a schematic cross-sectional diagram of a chip according to an embodiment of the present disclosure in a state where no electrical signal is applied; FIG. 3A is a schematic plane diagram of a chip according to an embodiment of the present disclosure in a state where an electrical signal is applied; and FIG. 3B is a schematic cross-sectional diagram of a chip according to an embodiment of the present disclosure in a state where an electrical signal is applied. The working principle of the chip 100 provided by the embodiments of the present disclosure is exemplarily described below with reference to FIGS. 2A-3B.

For example, in an example, as illustrated in FIG. 2A, a continuous phase A (such as a mineral oil) is injected by a positive pressure into the first inlet 141, a dispersed phase B (such as a PCR reaction solution) is injected by a positive pressure into the second inlet 142, nano-liter-level dividing and emulsification wrapping of the dispersed phase B are achieved by controlling the injection pressure and adopting appropriate sizes of the first channel 143 and the second channel 144, thereby forming a plurality of droplets 003 with a diameter of about 20 microns and having a uniform size, which enter the detection cavity 110 along with the continuous phase A. Here, no electrical signal is applied to the working electrode 120. The plurality of droplets 003 are randomly distributed in the detection cavity 110, i.e., the plurality of droplet 003 are irregularly distributed. As illustrated in FIG. 2B, the plurality of droplets 003 are also randomly distributed in a direction perpendicular to the first substrate 210 (that is, a second direction illustrated in the figure), and thus when the number of the droplets 003 is large, the plurality of droplets 003 may have problems such as overlapping and blocking. When the optical detection is performed, due to the overlapping and blocking of the droplet 003, the fluorescence generated by the droplets 003 may interfere with each other, and the positions of the plurality of droplets 003 in the second direction are not in the same horizontal plane, which also leads to a problem of inconsistent focusing surfaces, thereby increasing the difficulty of image processing, increasing the complexity and costs of the optical system used for detection, and reducing detection accuracy and detection efficiency.

As illustrated in FIG. 3A, an additionally provided control unit or control circuit may be used to apply an electrical signal to the working electrode 120 to control the plurality of droplets 003 to be regularly arranged. For example, the control unit or control circuit includes an alternating voltage source AC, which is electrically connected to the first electrode 121 and the second electrode 122 and is configured to output an alternating electrical signal to the first electrode 121 and the second electrode 122. For example, in an example, the alternating electrical signal is a sine alternating signal, for example, a continuously output analog sine waveform having a peak voltage of 500 mV and a frequency of 10 kHz. Certainly, the embodiments of the present disclosure are not limited thereto, and the alternating electrical signal may be other appropriate electrical signals, and the embodiments of the present disclosure have no limitation in this aspect.

Under the action of the alternating electrical signal, a non-uniform electric field is generated in the detection cavity 110. The droplet 003 is the PCR reaction solution containing movable charged ions such as magnesium (Mg) ions and chlorine (Cl) ions, and these charged ions are polarized under the action of the non-uniform electric field. Because the droplet 003 is an aqueous phase solution, the polarizability of the droplet 003 is different from the polarizability of the continuous phase A (the mineral oil) in the detection cavity 110, so that the droplet 003 is subjected to a dielectrophoresis force. The dielectrophoresis force can be expressed by following formula:

$${F}_{DEP} = π {a}^{3} {ɛ}_{m} Re [ \frac{{ɛ}_{P}^{*} - {ɛ}_{m}^{*}}{{ɛ}_{P}^{*} + 2 {ɛ}_{m}^{*}} ] ∇ {\vert E \vert}^{2} \approx π {a}^{3} {ɛ}_{m} Re [ \frac{{ɛ}_{P}^{*} - {ɛ}_{m}^{*}}{{ɛ}_{P}^{*} + 2 {ɛ}_{m}^{*}} ] \frac{{\vert E \vert}^{2}}{r} ,$$

where ε*=ε−jσ/ω, ε* represents the complex dielectric constant, ε represents the dielectric constant, σ represents the electrical conductivity, ω represents the angular frequency, P and m respectively represent the droplet and the surrounding medium (i.e., the mineral oil), a represents the semidiameter of the droplet, E represents the strength of the electric field, r represents the distance between the electrodes,

$$\frac{{ɛ}_{P}^{*} - {ɛ}_{m}^{*}}{{ɛ}_{P}^{*} + 2 {ɛ}_{m}^{*}}$$

represents the CM factor, the value of the CM factor ranges from 0.5 to 1, and the CM factor determines the direction of the dielectrophoresis force. The detailed description of the dielectrophoresis force may be referred to conventional design, which is not described in detail herein.

Under the action of the dielectrophoresis force, the droplet 003 moves against the static fluid viscous force and gathers and releases to a specific area. For example, the droplet 003 moves all the way to the edge of the first electrode 121 or the second electrode 122 (i.e., the edge of the first comb portion 1211 or the second comb portion 1221), and the spherical center of the droplet 003 overlaps with the edge of the first electrode 121 or the second electrode 122. It can be known through a simulation calculation that the edge of the first electrode 121 or the second electrode 122 is a point with the highest non-uniform electric field charge density, so the droplet 003 stays at this position. As illustrated in FIG. 3B, each droplet 003 is located at the edge of the first electrode 121 or the second electrode 122. Due to the spatial distribution of the non-uniform electric field, the dielectrophoresis force applied on the droplets 003 in the second direction drives all the droplets 003 to the same height (i.e., the same horizontal plane), thereby achieving three-dimensional control of the droplet position.

Therefore, by applying the alternating electrical signal to the working electrode 120, the plurality of droplets 003 are regularly arranged along the extending direction of the working electrode 120, thereby achieving three-dimensional control of the droplet position and reducing the complexity and costs of the optical system for detection, reducing the difficulty of image processing, and improving detection accuracy and detection efficiency.

For example, the chip 100 may realize integration of processes such as droplet generation and regular arrangement of the droplets. Through the cooperation with a peripheral equipment, the chip 100 may also realize the integration of all processes such as droplet generation, thermal cycle amplification, regular arrangement of the droplets, and optical detection. The chip 100 has a simple structure, does not require a complicated process, has low production costs, achieves miniaturization and integration, and reduces manual operations. The chip 100 may be applied not only in the digital PCR technology, but also in clinical and scientific research fields such as cell sorting control, protein detection, microbial detection, and the like.

It should be noted that, in the embodiments of the present disclosure, the plurality of droplets 003 may form a rectangular array, or may be arranged in other manner, as long as the plurality of droplets 003 have a certain regularity and are regularly arranged as a whole, and the specific arrangement of the droplets 003 is not limited in the embodiments of the present disclosure.

It should be noted that, in an example, when the alternating electrical signal is applied to the working electrode 120, the continuous phase A may be continuously injected into the first entry 141 by a positive pressure, and the continuous phase A passes through the detection cavity 110 and is discharged from the liquid outlet 132. In the detection cavity 110, the dielectrophoresis force applied to some droplets 003 is smaller than the fluid viscous force, and these droplets 003 are not driven to the edge of the first electrode 121 or the second electrode 122 and therefore are not captured. With the flowing of the continuous phase A, the uncaptured droplets 003 move along with the continuous phase A to the outside of the detection cavity 110, so that the droplets 003 remaining in the detection cavity 110 (i.e., the captured droplets 003) are regularly arranged, thereby reducing the complexity of image processing.

It should be noted that, in the embodiments of the present disclosure, the dispersed phase B injected through the second entry 142 may be a PCR reaction solution that has completed an amplification reaction, or may be a PCR reaction solution that has not been subjected to an amplification reaction. In a case where the dispersed phase B is a PCR reaction solution that has completed the amplification reaction, the dispersed phase B is divided into a plurality of droplets 003 and enters the detection cavity 110, and then, by applying the alternating electrical signal to the working electrode 120, the plurality of droplets 003 are regularly arranged for subsequent optical detection. In a case where the dispersed phase B is a PCR reaction solution that has not been subjected to the amplification reaction, the dispersed phase B is divided into a plurality of droplets 003 and enters the detection cavity 110, and then, a temperature control device additionally provided is required to heat and cool the detection cavity 110 to enable the DNA fragment in the droplets 003 to carry out the amplification reaction. For example, in some embodiments of the present disclosure, temperature control by a temperature control device using a Peltier effect may be implemented. After the amplification reaction is completed, the alternating electrical signal is applied to the working electrode 120, so that the plurality of droplets 003 are regularly arranged for subsequent optical detection.

FIG. 4 is a schematic cross-sectional diagram of another chip according to an embodiment of the present disclosure along an N-N′ direction in FIG. 1A. As illustrated in FIG. 4, a chip 200 is substantially the same as the chip 100 illustrated in FIGS. 1 to 3B except that the chip 200 further includes a passageway 240.

For example, the passageway 240 is disposed in the detection cavity 110 and is arranged along the extending direction of the working electrode 120. The passageway 240 is between the first substrate 210 and the second substrate 220. For example, the passageway 240 is used to provide a movement channel for the droplets 003, physically assist the regular arrangement of the droplets 003, and also play a supporting role. For example, the spacer layer 230 also defines the passageway 240 in the detection area 002, and a part of the spacer layer 230 defining the passageway 240 serves as a side wall 241 of the passageway 240. The side wall 241 may be formed in a form of, for example, a division wall. For example, the side wall 241 is located on the first comb portions 1211 and/or the second comb portions 1221, and the passageway 240 includes a space corresponding to edges, which are close to each other, of two adjacent first comb portions 1211, two adjacent second comb portions 1221, or a first comb portion 1211 and a second comb portion 1221 that are adjacent (for example, the space corresponding to the dotted block in FIG. 4).

Because the plurality of droplets 003 are regularly arranged along the extending direction of the working electrode 120 and are located at the edges of the first comb portions 1211 and/or the second comb portions 1221, the side wall 241 is disposed on the first comb portions 1211 and/or the second comb portions 1221, thereby preventing the side wall 241 from occupying the containing space for the droplets 003.

It should be noted that, in the embodiments of the present disclosure, the sizes of the chip 100 or 200 and each component are not limited, which may be determined according to actual requirements. For example, in an example, the chip 100 illustrated in FIG. 1A and FIG. 1B may be designed as follows. The chip 100 has a length L of 75 millimeters and a width L2 of 25 millimeters. A height h between the first substrate 210 and the second substrate 220 is 35 micrometers. The first entry 141 and the second entry 142 each have a diameter of 1.5 millimeters. The first comb portions 1211 and the second comb portions 1221 have a length of 40 millimeters, a width of 100 micrometers, and a pitch of 30 micrometers. The width of the first comb portions 1211 and the second comb portions 1221 is 100 micrometers. After the droplets are regularly arranged, there can be sufficient space in the detection cavity 110 for the droplets to move, so that the uncaptured droplets move to the outside of the detection cavity 110 along with the continuous phase A (such as the mineral oil), thereby reducing the complexity of image processing.

At least one embodiment of the present disclosure further provides a detection device, which includes the chip according to any one of the embodiments of the present disclosure, and further includes a control unit configured to apply an electrical signal to the working electrode. The detection device can arrange droplets regularly, realize three-dimensional control of droplet positions, reduce the complexity and costs of an optical system for detection, reduce the difficulty of image processing, improve detection accuracy and detection efficiency, achieve miniaturization and integration of detection chips, and reduce manual operations.

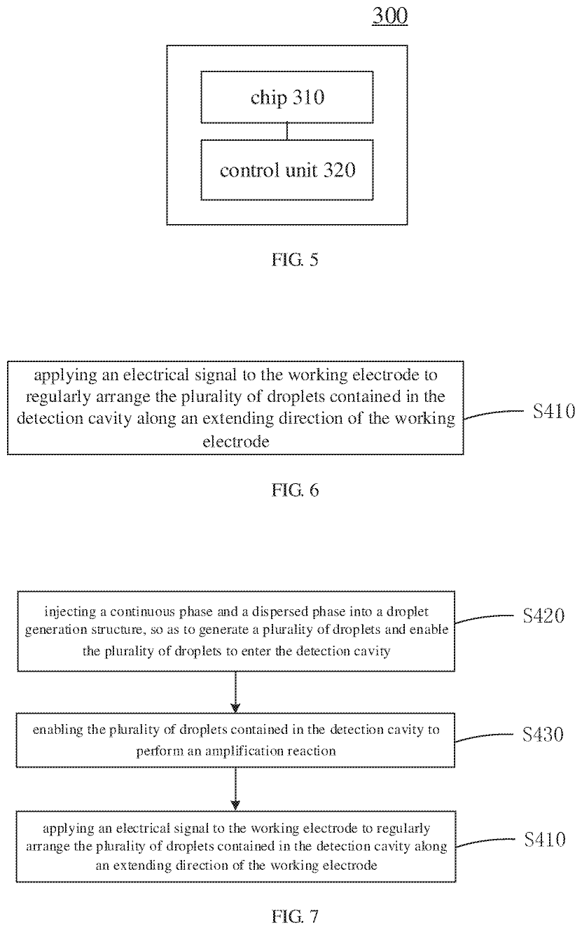

FIG. 5 is a schematic block diagram of a detection device according to an embodiment of the present disclosure. As illustrated in FIG. 5, a detection device 300 includes a chip 310 and a control unit 320. The chip 310 is the chip 100/200 according to any one of the embodiments of the present disclosure. The control unit 320 is electrically connected to the working electrode 120 of the chip 310, and is configured to apply an electrical signal to the working electrode 120 to control a plurality of droplets to be regularly arranged. The detection device 300 may be a PCR amplification system or a PCR detection system, and may also be other appropriate devices, which is not limited in the embodiments of the present disclosure.

For example, the electrical signal applied, by the control unit 320, to the working electrode 120 is an alternating electrical signal, so that a non-uniform electric field can be generated in the detection cavity 110 to enable a plurality of droplets to move and be regularly arranged under the action of a dielectrophoresis force. For example, the alternating electrical signal may be a sine alternating signal or other appropriate alternating signals, which is not limited in the embodiments of the present disclosure.

For example, the control unit 320 may be implemented as a dedicated or general-purpose electronic hardware (or circuit), such as a single-chip microcomputer or the like, which is not limited in the embodiments of the present disclosure. The specific structure of the electronic hardware is not limited, and may include an analog device, a digital chip, or other appropriate devices. It should be noted that in some embodiments of the present disclosure, the detection device 300 may further include more or fewer components, for example, the detection device 300 may further include an optical detection unit or system, which may be determined according to actual requirements, and the embodiments of the present disclosure have no limitation in this aspect. Regarding the technical effects of the detection device 300, reference may be made to the above description of the chip 100/200, which are not repeated herein.

At least one embodiment of the present disclosure further provides a method of operating the chip according to any one of the embodiments of the present disclosure, and by adopting the method to operate the chip according to the embodiments of the present disclosure, it is possible to arrange droplets in the chip regularly, realize three-dimensional control of droplet positions, reduce the complexity and costs of an optical system for detection, reduce the difficulty of image processing, improve detection accuracy and detection efficiency, achieve miniaturization and integration of detection chips, and reduce manual operations.

FIG. 6 is a schematic flow chart of a method of operating a chip according to an embodiment of the present disclosure. As illustrated in FIG. 6, in an example, the method of operating the chip includes following operations.

Step S410: applying an electrical signal to the working electrode 120 to regularly arrange the plurality of droplets 003 contained in the detection cavity 110 along an extending direction of the working electrode 120.

FIG. 7 is a schematic flow chart of another method of operating a chip according to an embodiment of the present disclosure. As illustrated in FIG. 7, in another example, the method of operating the chip includes following operations.

Step S420: injecting the continuous phase A and the dispersed phase B into the droplet generation structure 140 to generate the plurality of droplets 003, and enabling the plurality of droplets 003 to enter the detection cavity 110.

Step S430: enabling the plurality of droplets 003 contained in the detection cavity 110 to perform the amplification reaction.

Step S410: applying an electrical signal to the working electrode 120 to regularly arrange the plurality of droplets 003 contained in the detection cavity 110 along an extending direction of the working electrode 120.

For example, the first entry 141 of the droplet generation structure 140 is configured to receive the continuous phase A, and the second entry 142 of the droplet generation structure 140 is configured to receive the discrete phase B. For example, the continuous phase A includes a mineral oil, and the dispersed phase B includes a PCR reaction solution (i.e., a sample solution). For example, the continuous phase A and the dispersed phase B may be injected by using an air pressure injection method. Certainly, the embodiments of the present disclosure are not limited to this case, and the continuous phase A and the dispersed phase B may also be injected by using other appropriate methods such as using an injection pump or a peristaltic pump.

The specific process of the method is exemplarily described below.

First, nucleic acid extraction is performed, that is, DNA fragments is extracted, 1 mL of a test sample is taken, 50 mL of lysate is added, and repeatedly drawing and injecting with a pipette is performed, the supernatant is taken after the deposited sample is fully dispersed by a shaker, and DNA fragments are obtained by using the spin column method, i.e., DNA amplification templates are obtained, and then the DNA fragments are left to stand. The amplification reagent is taken out 30 minutes in advance and is stood at room temperature for thawing. A reaction system solution, a primer probe mixture (including a specific sequence primer, a probe dNTP, a magnesium ion solution, etc.), a human cytomegalovirus enzyme (HCMV enzyme) mixture (including a Tap enzyme and a UDG enzyme), DNA amplification templates is mixed at a certain ratio and then shaken by using a pipette, so as to obtain a PCR reaction solution.

Then, the fluorocarbon oil containing a specific additive (for example, poly-carboxylates) is injected into the chip 100 through the first entry 141 by a positive pressure. After the fluorocarbon oil fills the detection cavity 110, the PCR reaction solution is injected into the chip 100 through the second entry 142 by a positive pressure, and the fluorocarbon oil is continuously injected into the chip 100 through the first entry 141 by a positive pressure. As a result, droplets of the PCR reaction solution start to be generated. After the droplets fills the detection cavity 110, the positive pressure injection is stopped, and the chip 100 is sealed to perform a temperature cycle amplification process. For example, the temperature of the detection cavity 110 may be raised and lowered by a temperature control device additionally provided, so that the droplets undergo an amplification reaction.

After the amplification is completed, an alternating electrical signal s applied to the working electrode 120 by the control unit 320 to generate a non-uniform electric field in the detection cavity 110. The droplets move under the action of the dielectrophoresis force, gather along the extending direction of the working electrode 120, and stay at the edges of the first comb portions 1211 and the second comb portions 1221, thereby realizing the regular arrangement of the droplets. The uncaptured droplets continue to move along the working electrode 120 under the action of the viscous force of the fluorocarbon oil until they exit the detection cavity 110 through the liquid outlet 132.

After the droplet array is stabilized, another optical detection device or system, such as a chip scanning system, is used to scan and record the fluorescence signal and the blank signal line by line. For example, the fluorescence signal appears as a peak. Finally, the initial concentration of the sample is calculated by using the poisson distribution to achieve absolute quantitative detection of the nucleic acids.

It should be noted that the specific process of the above operation method is only exemplary and not limiting, and when using the chip provided by the embodiments of the present disclosure for a PCR detection, operation processes meeting actual requirements may be determined according to factors such as the actual operating environment, the adopted equipment, chemicals, etc.

Regarding the details and technical effects of the method, reference may be made to the above description of the chip 100/200 and the detection device 300, which are not repeated herein.

The following statements should be noted.

(1) The accompanying drawings involve only the structure(s) in connection with the embodiment(s) of the present disclosure, and other structure(s) can be referred to common design(s).

(2) In case of no conflict, the embodiments of the present disclosure and the features in the embodiments can be combined with each other to obtain new embodiments.

The foregoing merely are exemplary embodiments of the disclosure, and not intended to define the scope of the disclosure, and the scope of the disclosure is determined by the appended claims.

1. A chip, comprising:
a detection cavity, configured to be capable of containing a plurality of droplets; and
a working electrode, arranged in the detection cavity and configured to regularly arrange the plurality of droplets in the detection cavity along an extending direction of the working electrode.

2. The chip according to claim 1, wherein the working electrode comprises a first electrode and a second electrode,
the first electrode comprises a plurality of first comb portions, the second electrode comprises a plurality of second comb portions, and the plurality of first comb portions of the first electrode and the plurality of second comb portions of the second electrode are arranged in an interlaced manner.

3. The chip according to claim 2, wherein each of the plurality of first comb portions and the plurality of second comb portions is of a straight line shape, a polyline shape, or a curve shape.

4. The chip according to claim 2 or 3, wherein the plurality of first comb portions and the plurality of second comb portions are arranged side by side and are equally spaced.

5. The chip according to claim 2, wherein the detection cavity comprises a liquid inlet and a liquid outlet, and the liquid inlet and the liquid outlet are at opposite ends of the detection cavity in the extending direction of the working electrode.

6. The chip according to claim 5, further comprising a droplet generation structure,
wherein the droplet generation structure is connected with the liquid inlet, and is configured to generate the plurality of droplets and input the plurality of droplets into the detection cavity through the liquid inlet.

7. The chip according to claim 6, wherein the droplet generation structure comprises a first entry, a second entry, a first channel, a second channel, and a cross connection portion,
the first entry is configured to be in communication with the first channel, the second entry is configured to be in communication with the second channel, the first channel and the second channel are connected through the cross connection portion, and
the cross connection portion is connected with the liquid inlet.

8. The chip according to claim 6 or 7, further comprising a first substrate, a second substrate, and a spacer layer,
wherein the chip further comprises an injection area and a detection area, the spacer layer defines the droplet generation structure in the injection area, and the spacer layer defines the detection cavity in the detection area.

9. The chip according to claim 8, wherein the working electrode is on a side of the first substrate close to the second substrate or on a side of the second substrate close to the first substrate.

10. The chip according to claim 8, wherein at least one of the first substrate and the second substrate is a transparent substrate.

11. The chip according to claim 8, further comprising a passageway, wherein the passageway is in the detection cavity and arranged along the extending direction of the working electrode.

12. The chip according to claim 11, wherein the spacer layer further defines the passageway in the detection area, a part of the spacer layer defining the passageway serves as a side wall of the passageway,
the side wall is on the first comb portions and/or the second comb portions, and the passageway comprises a space corresponding to edges, which are close to each other, of two adjacent first comb portions, two adjacent second comb portions, or a first comb portion and a second comb portion that are adjacent.

13. A detection device, comprising the chip according to claim 1, and further comprising a control unit, wherein the control unit is configured to apply an electrical signal to the working electrode.

14. The detection device according to claim 13, wherein the electrical signal comprises an alternating electrical signal.

15. A method of operating the chip according to claim 1, comprising:
applying an electrical signal to the working electrode to regularly arrange the plurality of droplets contained in the detection cavity along the extending direction of the working electrode.

16. The method of operating the chip according to claim 15, further comprising:
injecting a continuous phase and a dispersed phase into a droplet generation structure, so as to generate the plurality of droplets and enable the plurality of droplets to enter the detection cavity.

17. The method of operating the chip according to claim 16, wherein a first entry of the droplet generation structure is configured to receive the continuous phase, and a second entry of the droplet generation structure is configured to receive the dispersed phase.

18. The method of operating the chip according to claim 16, wherein the continuous phase comprises a mineral oil, and the dispersed phase comprises a sample solution.

19. The method of operating the chip according to claim 16, wherein the continuous phase and the dispersed phase are injected by using an air pressure injection method.

20. The method of operating the chip according to claim 15, further comprising:
enabling the plurality of droplets contained in the detection cavity to perform an amplification reaction.
